(19) United States
(12) Patent Application Publication
(10) Pub. No.: US 2006/0287668 A1
(43) Pub. Date: Dec. 21, 2006

(54) Apparatus and methods for intravascular embolic protection

(76) Inventors: Natalie V. Fawzi, Belmont, CA (US); Dwight P. Morejohn, Davis, CA (US); Amr Salahieh, Saratoga, CA (US)

Correspondence Address: WILSON SONSINI GOODRICH & ROSATI, 650 PAGE MILL ROAD, PALO ALTO, CA, 94304-1050, US

(21) Appl. No.: 11/155,309

(22) Filed: Jun. 16, 2005

(51) Int. Cl. A61M 29/00

(52) U.S. Cl. 606/200

(57) ABSTRACT

The present invention provides intravascular embolic protection apparatus including a blood filter element having an accommodating passageway adapted to permit passage of a procedure device therethrough and to substantially seal against passage of particles between the embolic protection apparatus and the procedure device by accommodating to a size and shape of the procedure device. Furthermore, the present invention provides a method of performing an endovascular procedure on a patient including the steps of delivering an embolic protection apparatus to a location within a vascular lumen of the patient; passing a procedure device through an accommodating passageway of the apparatus, the accommodating passageway accommodating to a size and shape of the procedure device; performing the endovascular procedure; and removing the procedure device from the patient.

### BACKGROUND OF THE INVENTION

The present invention relates to methods and apparatus for protecting a patient from embolization during an endovascular procedure, for example, during a retrograde endovascular procedure, such as valvuloplasty or endovascular replacement of the patient's heart valve.

In many endovascular procedures, a procedure device is advanced intravascularly in an antegrade fashion (with the direction of blood flow) to a treatment site where the endovascular procedure is performed with the procedure device. Some procedures, such as carotid stenting, may release embolic material into the patient's bloodstream. Embolic filters and diverters have been developed to filter or route dangerous emboli released into the blood, such that the emboli do not travel to the cerebral vasculature and/or do not form a blood clot.

In antegrade procedures, the embolic filter is commonly placed downstream and distal of the treatment site prior to performance of the endovascular procedure. The procedure device then is advanced to the treatment site proximal and upstream of the filter, and the procedure is performed. The embolic filter removes or diverts emboli generated during or caused by the procedure. Here and throughout this specification, distal refers to a position further from the user as measured along the path of the system while proximal refers to the position closer to the user as measured along the path of the system.

Embolic protection also may be desirable in procedures where the procedure device is advanced in a retrograde fashion (against the direction of blood flow) to the treatment site. In these procedures, it would be desirable to provide embolic protection proximal of the treatment site, which is downstream of the direction of blood flow in retrograde procedures. However, since the embolic filter typically seals against a wall of a blood vessel, many known filters are not suitable for retrograde use in combination with a procedure device because the procedure device cannot be advanced across the filter distal and upstream to the treatment site.

In recent years, advancements in minimally invasive surgery and interventional cardiology have encouraged some investigators to pursue percutaneous, endovascular replacement of the aortic heart valve. See, e.g., U.S. Pat. No. 6,168,614, which is incorporated herein by reference in its entirety. The replacement valve may be delivered in a retrograde fashion and deployed across the native diseased valve to permanently hold the native valve open, thereby alleviating a need to excise the native valve and to surgically position the replacement valve in place. Optionally, a valvuloplasty may be performed prior to, or after, deployment of the replacement valve.

Since the native valve may be calcified or stenosed, valvuloplasty and/or deployment of the replacement valve poses a risk of loosening and releasing embolic material into the patient's blood stream. This material may, for example, travel downstream (proximally) through the patient's aorta and carotid arteries to the cerebral vasculature of the brain. Thus, a risk exists of reduction in mental faculties, stroke or even death during endovascular heart valve replacement, due to release of embolic material.

In view of the foregoing, it would be desirable to provide methods and apparatus for protecting against embolization, for example, during retrograde endovascular procedures.

### SUMMARY OF THE INVENTION

One aspect of the invention provides an intravascular embolic protection apparatus including: a blood filter element adapted to capture particles and to allow blood to flow therethrough; an opening adapted to face blood flow; a closed portion adapted to retain captured particles; and an accommodating passageway adapted to permit passage of a procedure device therethrough from a position proximal to the closed portion to a position distal to the opening and to substantially seal against the passage of particles between the embolic protection apparatus and the procedure device by accommodating to a size and shape of the procedure device.

Another aspect of the invention provides a method of performing an endovascular procedure on a patient with a procedure device, including the steps of: delivering an embolic protection apparatus to a location within a vascular lumen of the patient, the embolic protection apparatus comprising an accommodating passageway; passing the procedure device through the accommodating passageway from a point proximal to the embolic protection apparatus to a point distal to the embolic protection apparatus after the delivering step, the accommodating passageway accommodating to a size and shape of the procedure device; performing the endovascular procedure; and removing the procedure device from the patient.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B are an isometric schematic view and a schematic detail view of an embodiment of intravascular embolic protection apparatus.

FIGS. 2A and 2B are schematic detail views of the blood filter element of the apparatus of FIG. 1, illustrating an accommodating passageway of the blood filter element.

FIGS. 3A and 3B is a schematic detail side and side-sectional views of the blood filter element of the apparatus of FIG. 1. FIGS. 3A and 3B illustrate alternative material construction configurations of the apparatus in FIG. 1. FIG. 3A additionally illustrates accommodation of the passageway to a size and shape of a device passed through the passageway, and the accommodating passageway substantially sealing against a guidewire.

FIGS. 4A-4C are side views, partially in section, of the intravascular embolic protection apparatus of FIGS. 1-3 disposed in a reduced delivery configuration.

FIGS. 5A-5C are side views and cross-sectional views of another embodiment of the intravascular embolic protection apparatus disposed in a reduced delivery configuration.

FIGS. 6A-6G are side-sectional views illustrating a method of using the embolic protection apparatus of FIGS. 1-4 in combination with a procedure device.

FIG. 7 is a schematic side view illustrating a method of using the embolic protection apparatus to protect against embolization in combination with a procedure device for performing an endovascular heart valve replacement.

FIG. 8 is a schematic side view, partially in section, illustrating another method of using the embolic protection apparatus to protect against embolization during endovascular heart valve replacement.

FIGS. 9A and 9B are schematic side and end views illustrating an embodiment of the embolic protection apparatus comprising an alternative recapture guide element.

FIG. 10 is a side view, partially in section, illustrating a method of collapsing the embolic protection apparatus of FIG. 9 for retrieval or recapture providing improved retention of emboli.

FIGS. 11A and 11B are a schematic side view, partially in section, and an isometric schematic detail view of an embodiment of the embolic protection apparatus comprising a capture tool for recapturing the embolic protection apparatus.

FIG. 12 is a schematic side view, partially in section, of an embodiment of the embolic protection apparatus comprising an alternative recapture tool providing improved retention of emboli.

FIGS. 13A and 13B are schematic side views of an embodiment of the embolic protection apparatus comprising another alternative recapture tool providing improved retention of emboli.

FIGS. 14A-14B are schematic side views of an embodiment of the embolic protection apparatus comprising a recapture tool in combination with a cinch mechanism for retaining captured particles within the embolic protection apparatus.

FIGS. 15A-15C are schematic views of embodiments of the embolic protection apparatus having passageways positioned at different locations along a diameter of the apparatus.

FIGS. 16A-16J are schematic side-sectional and isometric views of alternative embodiments of the embolic protection apparatus.

FIGS. 17A-17C are schematic side and cross-sectional views of an alternative embodiment of the embolic protection apparatus having a passageway comprising a fold in the apparatus.

### DETAILED DESCRIPTION OF THE INVENTION

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to methods and apparatus for protecting a patient from embolization during an endovascular procedure, for example, during a retrograde endovascular procedure, such as valvuloplasty or endovascular replacement of the patient's heart valve. More particularly, the present invention relates to methods and apparatus for providing embolic protection by filtering blood downstream of the endovascular procedure during the procedure. Applicant has previously described methods and apparatus for protecting against embolization during retrograde endovascular replacement of a patient's diseased heart valve, for example, in co-pending U.S. patent application Ser. No. 10/920,736, filed Aug. 17, 2004, which is incorporated herein by reference in its entirety.

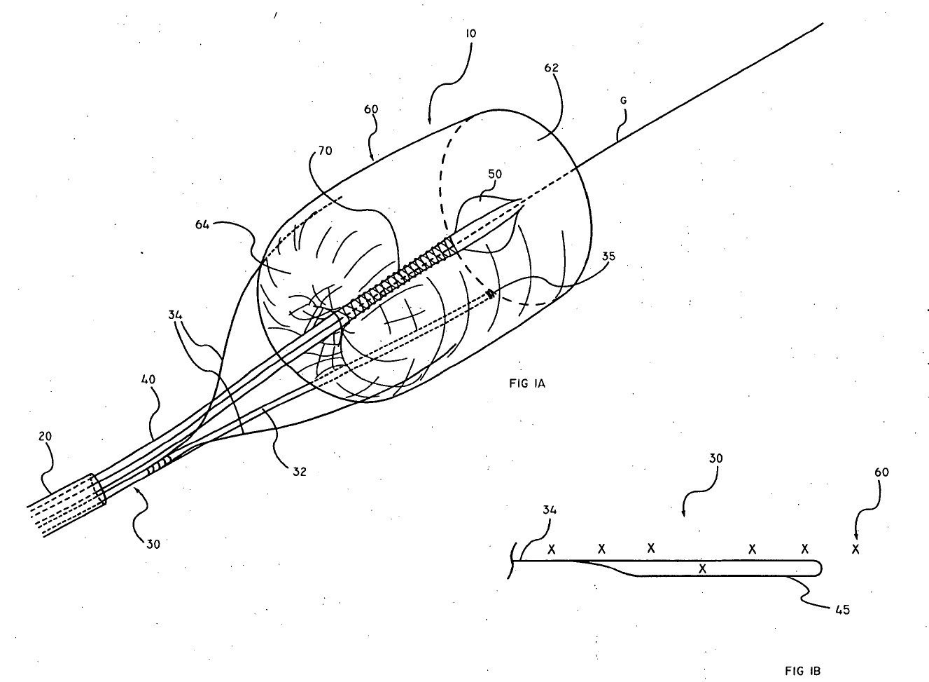

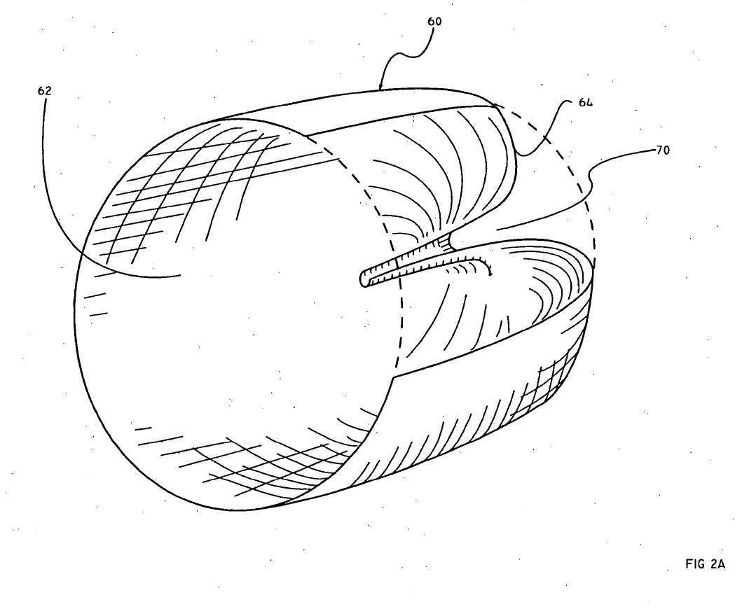

With reference to FIG. 1, a first embodiment of intravascular embolic protection apparatus of the present invention is described. As seen in FIG. 1A, embolic protection apparatus 10 comprises delivery sheath or catheter 20, blood filter attachment element 30, guidewire tube 40 through which guidewire G may pass, nosecone 50 and blood filter element 60. Blood filter element 60 is adapted to capture particles and to allow blood to flow through the filter element. The blood filter element may, for example, comprise a finely woven mesh of a single wire or multiple wires, or a composite of a mesh and filter material.

Attachment element 30 comprises elongated member 32 that is coupled for example, at a distal attachment point 35 as depicted in FIG. 1 to blood filter element 60 for anchoring or maintaining a position of the filter element, and that extends proximally to a proximal region of apparatus 10 for manipulation by a medical practitioner.

Attachment element 30 may additionally be affixed to the patient at the proximal end by the medical practitioner, thereby anchoring the blood filter element 60, and may be manipulated to effect recapture of the blood filter element 60. The attachment element further comprises attachment wires 34 that extend from elongated member 32 and interface with blood filter element 60, for example, at points more proximal than the attachment point 35 of elongated member 32 to the filter element.

FIG. 1B illustrates an example of a technique for interfacing attachment wires 34 and/or elongated element 32 to blood filter element 60 to attach element 30 to the filter element. As seen in FIG. 1B, elongated member 34 may comprise a distal loop or eyelet attachment 45 that captures crossing filaments or wires (shown as X's in the figure) of the braid or mesh that forms blood filter element 60 to attach to the filter element. Additionally, eyelet 45 can be lengthened to allow for a sliding interface between the attachment member and the filter element. Alternatively, wires 34 may be interwoven with blood filter element 60 such that they do not capture blood filter element 60 but slide through (not shown). Additional alternative attachment wire interfaces will be apparent.

Attachment wires 34 may serve as recapture guide elements that facilitate sheathing or recapturing of blood filter element 60 within catheter 20 or within another catheter after filtering during an endovascular procedure. Wires 34 illustratively comprise longitudinal recapture wires, but other shaped wires, such as spiral capture wires described hereinafter, alternatively or additionally may be provided.

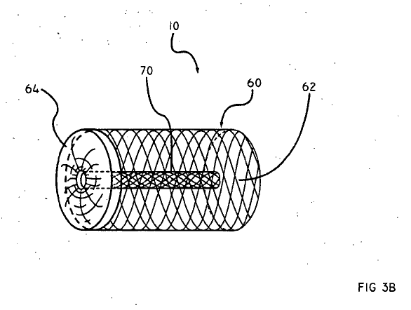

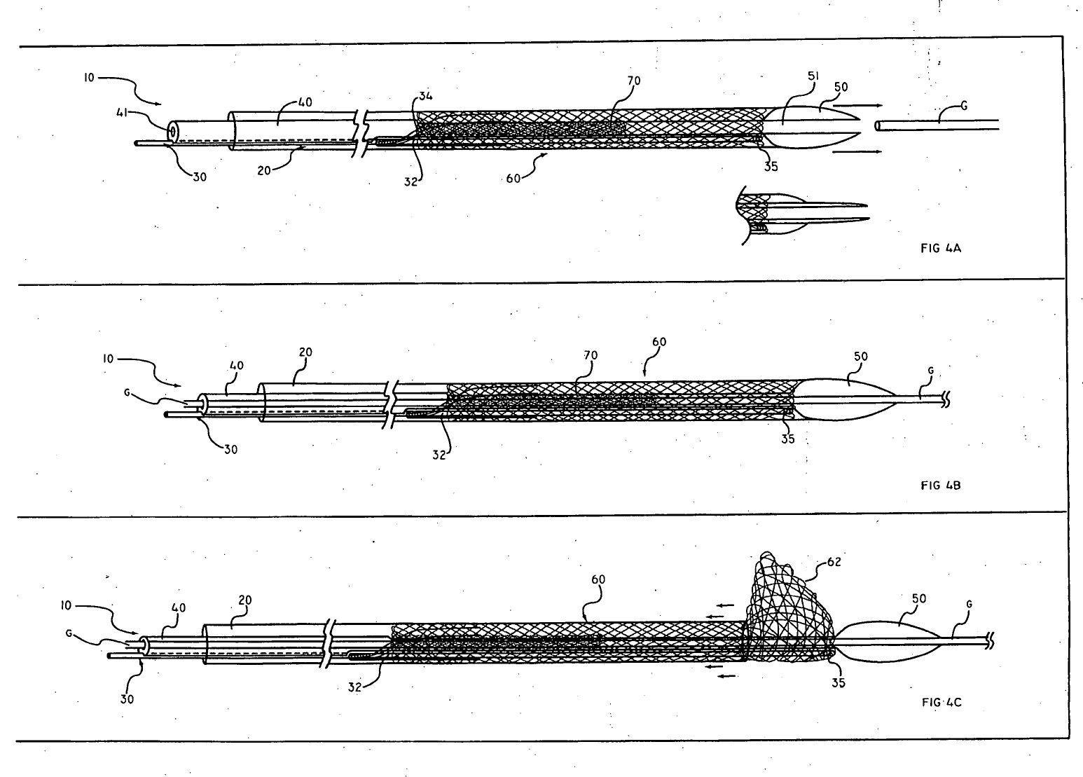

Filter element 60 comprises opening 62, closed portion 64 and accommodating passageway 70. Opening 62 is disposed at a distal region of the filter element and is adapted to face blood flow. Closed portion 64 is located more proximally along the filter element and is adapted to retain captured particles. Guidewire tube 40 facilitates advancement of the system over guidewire G in the reduced delivery configuration illustrated hereafter in FIG. 4. Guidewire tube 40 can be removed from accommodating passageway 70 and replaced with a procedure device that is advanced over guidewire G and through the accommodating passageway. The guidewire tube can be replaced during the procedure when the procedure device is not in place. In FIG. 1, a nosecone 50 illustratively is coupled to guidewire tube 40. As described hereinafter, a nosecone alternatively may be coupled to blood filter attachment element 30, for example, distal of blood filter element 60.

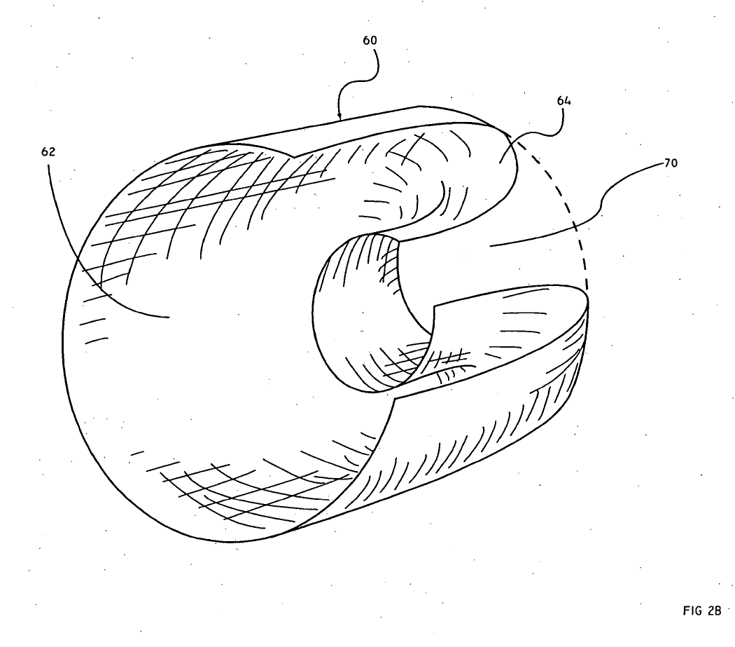

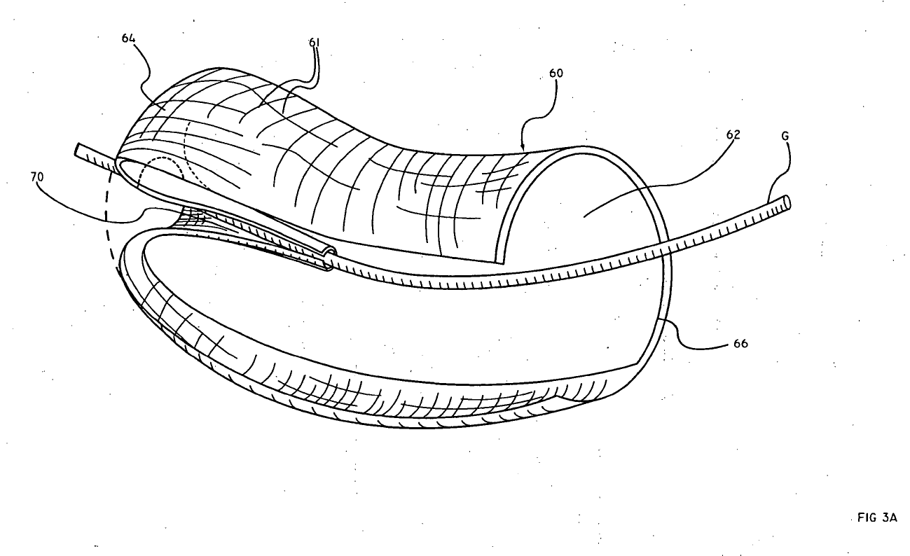

Referring now to FIGS. 2 and 3, in combination with FIG. 1, accommodating passageway 70 comprises a lumen through blood filter element 60 that is adapted to permit passage of a procedure device through the passageway from a position proximal to closed portion 64 to a position distal to opening 62, and to substantially seal against passage of particles between embolic protection apparatus 10 and the procedure device by accommodating to the size and shape of the procedure device. Furthermore, as seen in FIG. 2A, the passageway seals by closing to a diameter small enough to substantially prevent passage of particles through the passageway when a procedure device is not disposed in the passageway. The passageway preferably is self-sealing and is biased toward the sealed position. The passageway may, for example, comprise an inverted section of filter element 60, as shown in FIGS. 2 and 3.

As a procedure device passes through passageway 70, the passageway expands to accommodate the size and shape of the procedure device, as seen in FIG. 2B. Passageway 70 is expandable to permit devices of different sizes to pass therethrough. The passageway may, for example, be configured to accommodate procedure devices having a diameter of up to about 24 Fr, though this diameter should in no way be construed as limiting. Additionally, the use of the accommodating passageway allows the use of accessory devices to the primary procedure device, such as an introducer sheath. For example, the embolic protection apparatus can be deployed for use with an introducer sheath such that the introducer sheath is advanced through the accommodating passageway and the primary procedure device is thereafter inserted through the sheath.

In FIG. 3A, as the guidewire G passes through passageway 70, the self-sealing passageway 70 is biased toward the sealed position against the guidewire. Passageway 70 illustratively comprises an opening that tapers from the proximal region of the passageway to the distal region. This taper may provide guidance and facilitate passage of procedure devices through the passageway.

Blood filter element 60 may be configured for self-expansion from a reduced delivery configuration within sheath 20 to the expanded deployed configuration of FIG. 1. Additionally, blood filter element 60 may be configured to conform to the space within which it is deployed. As illustrated in FIGS. 2 and 3, passageway 70 may be expandable independent of the rest of blood filter element 60. Blood filter element 60 may be radially symmetrical as shown, or may comprise an alternative geometry, such as a bilateral symmetry, as described hereinafter. Furthermore, passageway 70 may be positioned in the center of blood filter element 60 as shown, or may be positioned off-center, as described hereinafter. Closed portion 64 of blood filter element 60 may comprise a taper that facilitates recapture of the blood filter element after an endovascular procedure, as described hereinafter. The filter element may, for example, be recaptured within a retrieval catheter or sheath. The taper of closed portion 64 may be radially symmetric as shown, or may comprise any other desired profile.

Blood filter element 60 illustratively comprises mesh material 61 that has been formed into a tube having an inverted, tapered end that defines passageway 70 and closed portion 64. Opening 62 and closed portion 64 of the blood filter element surround passageway 70. Mesh material 61 and/or blood filter element 60 provide a bias force that substantially seals the passageway; the bias force may be overcome to permit passage of a procedure device through the passageway.

The mesh material of blood filter element 60 may comprise a self-expanding mesh, for example, a mesh formed from a self-expanding material such as Nitinol or spring steel, or may comprise a mesh woven in a manner facilitating self-expansion.

Mesh material 61 may, for example, be formed from a single wire, from multiple wires and/or from multiple meshes. The mesh material may, for example, be heat-set in the configuration of FIG. 1. The mesh material optionally may be covered at least in part by filter material 66, which may comprise a material of known porosity. The porosity may, for example, be specified to allow for passage of blood therethrough while capturing embolic particles within the blood filter element.

With reference to FIG. 3B, blood filter element 60 of apparatus 10 illustratively comprises a multiple piece construction where the self expanding passageway 70 comprises a mesh material 61 formed in a tubular configuration which is attached to a third element which forms the closed proximal portion 64 to form the blood filter element 60. The closed portion 64 illustratively comprises a filter material which is fixed to the proximal edges of both the passageway 70 and the filter element 60.

With reference now to FIG. 4, blood filter element 60 of apparatus 10 is configured for delivery via sheath or catheter 20. As seen in FIG. 4A, blood filter element 60 may be disposed in a reduced delivery profile within sheath 20. Guidewire G may be percutaneously advanced to a treatment site using, for example, well-known percutaneous techniques, and apparatus 10 then may be advanced over the guidewire. Nosecone 50 comprises lumen 51 that is contiguous with lumen 41 of guidewire tube 40. As seen in FIG. 4B, guidewire G may be inserted through nosecone lumen 51 and through guidewire tube lumen 41, and apparatus 10 may then be advanced over the guidewire into position, for example, proximal and downstream of the treatment site.

Once properly positioned, catheter 20 may be retracted while attachment element 30, and thereby blood filter element 60, is held stationary. As seen in FIG. 4C, retraction of the catheter causes the blood filter element to self-expand. The filter element may be configured to expand asymmetrically as shown. This asymmetry during expansion may be helpful when positioning catheter 20. Continued retraction of the catheter causes the blood filter element to expand to the fully deployed configuration of FIG. 1. If repositioning is desired, the filter can be recaptured and repositioned any time during the procedure. Guidewire tube 40 and nosecone 50 then may be removed through passageway 70, and a procedure device may be advanced over guidewire G and through the passageway to perform an endovascular procedure.

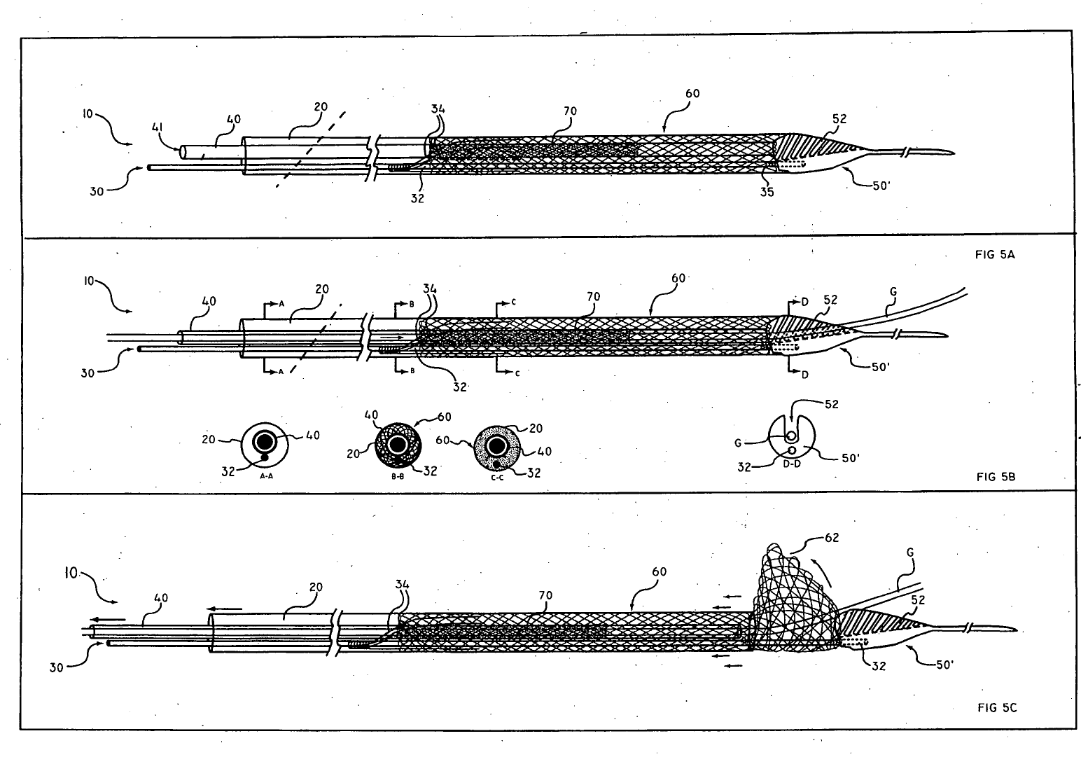

With reference to FIG. 5, another embodiment of the reduced delivery configuration of apparatus 10 is described. As seen in FIG. 5A, nosecone 50′ illustratively is coupled to elongated member 32 of blood filter attachment element 30 distal of blood filter element 60. As seen in cross-section D-D of FIG. 5B, nosecone 50′ comprises notch or cut-out 52 that facilitates passage of guidewire G out of the nosecone. In FIG. 5B, apparatus 10 is advanced over the guidewire. In FIG. 5C, catheter 20 is retracted while attachment element 30 and the guidewire is held stationary, which causes filter element 60 to self-expand.

Since the nosecone is not attached to guidewire tube 40, the guidewire tube optionally may be retracted simultaneously with catheter 20. Alternatively, the guidewire tube may be retracted after expansion of the filter element. Guidewire G exits nosecone 50′ through notch 52 as the filter element expands. In contrast to the embodiment of FIG. 4, nosecone 50′ remains distal of filter element 60 during passage of a procedure device through passageway 70 and during the endovascular procedure.

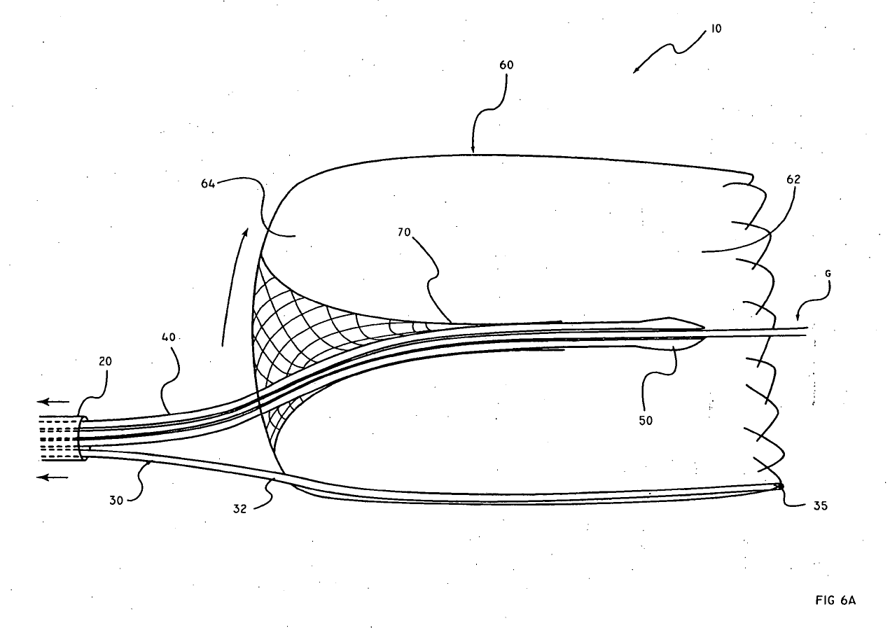

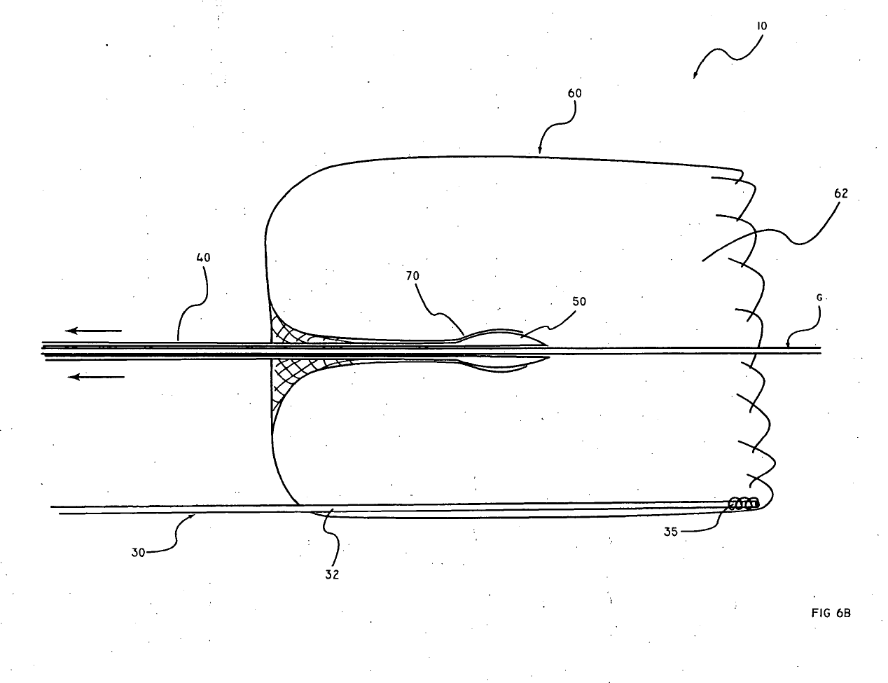

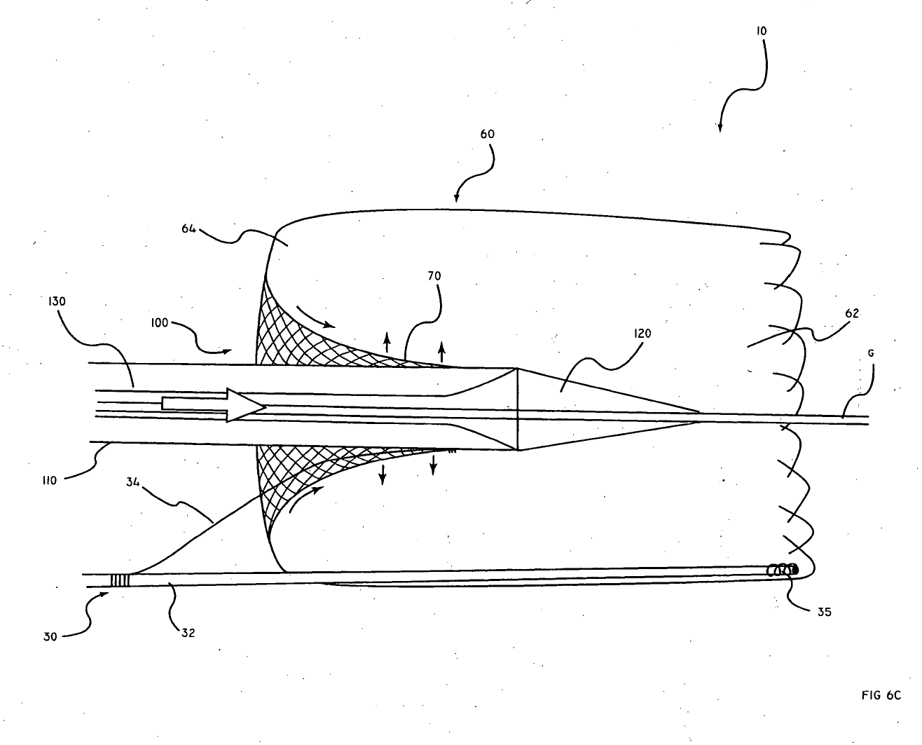

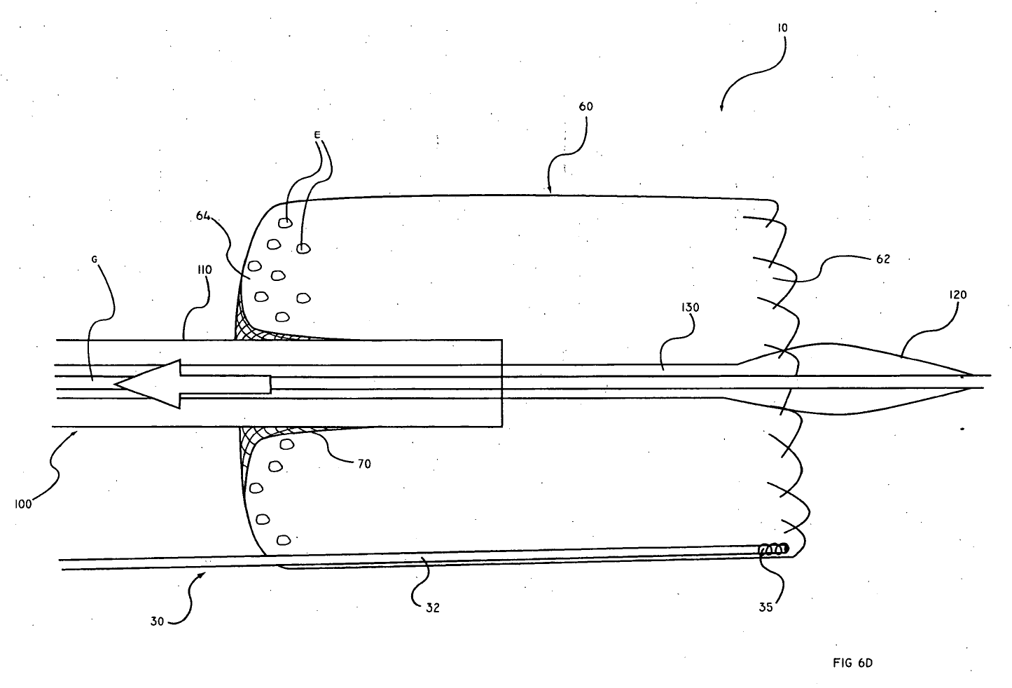

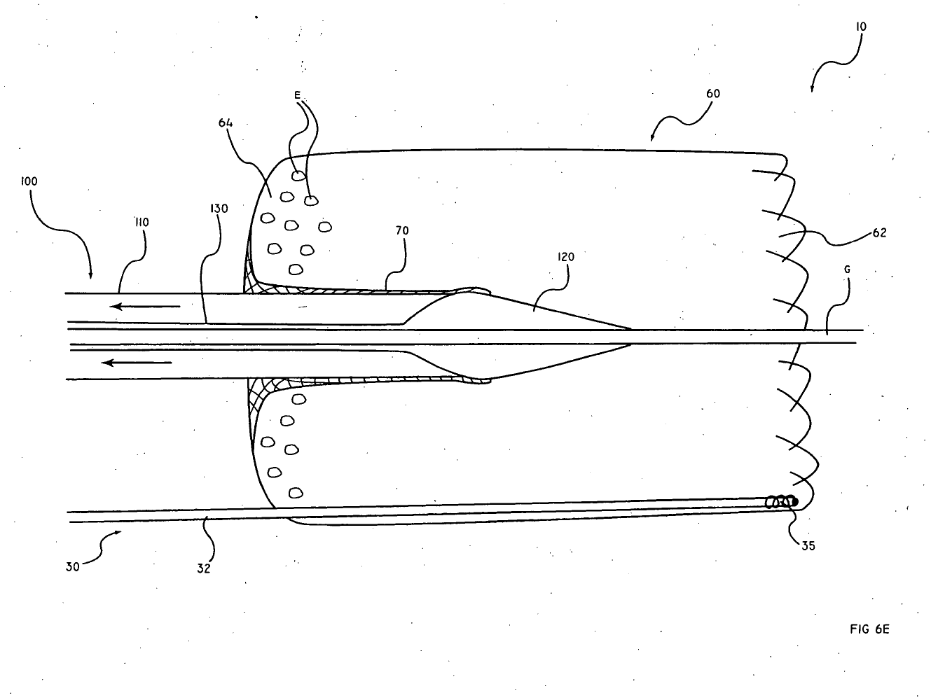

With reference now to FIGS. 6, a method of providing embolic protection with the apparatus of FIGS. 1-4, while performing an endovascular procedure on a patient with a procedure device is described. As seen in FIG. 6A, embolic protection apparatus 10 has been delivered to a location within a vascular lumen of the patient, catheter 20 has been retracted, and blood filter 60 is expanded as described with respect to FIGS. 4. The apparatus may, for example, be advanced in a retrograde fashion, such that opening 62 of blood filter element 60 faces a direction of blood flow.

As seen in FIG. 6B, guidewire tube 40 is retracted relative to attachment element 30 and blood filter element 60, which removes the guidewire tube and nosecone 50 through passageway 70, leaving the guidewire in place. Passageway 70 accommodates the size and shape of the guidewire tube and nosecone as they pass proximally (out) through the passageway. As seen in FIG. 6C, once the guidewire tube and nosecone have been removed, procedure device 100, which may, for example, comprise apparatus for endovascular replacement of the patient's heart valve comprising its own nosecone, is passed over the guidewire and through the accommodating passageway from a point proximal to filter element 60 to a point distal to the filter element. Passageway 70 comprises a sealing lumen in the filter element through which procedure device 100 is passed.

The accommodating passageway adapts to a size and shape of the procedure device. Passing the procedure device through the passageway comprises opening the passageway with the procedure device by overcoming the passageway's sealing bias. Passageway 70 self-seals against procedure device 100 when the device is passed through the passageway.

Next, an endovascular procedure is performed with the procedure device. During the endovascular procedure, an implant, such as an endovascular replacement heart valve, may, for example, be delivered from the annular space between central shaft 130 and catheter sheath 110 of procedure device 100. In such an embodiment of the procedure device, sheath 110 may be retracted relative to shaft 130 at the treatment site for deployment of the replacement valve implant.

If emboli E are generated during the endovascular procedure, the emboli are carried downstream and are filtered from the patient's blood by blood filter element 60. The emboli accumulate and/or are captured within closed portion 64 of the filter element. Procedure device 100 then may be removed from the patient.

As seen in FIG. 6D, during removal of procedure device 100, catheter sheath 110 may, for example, be partially or fully removed from apparatus 10 and from the patient independent of shaft 130 and nosecone 120, which is coupled to the shaft. Then, nosecone 120 and central shaft 130 may be retracted and removed.

In FIG. 6E, although nosecone 120 and sheath 110 illustratively are approximated within passageway 70, it should be understood that the nosecone and sheath alternatively may be approximated distal of the passageway and/or of blood filter element 60, or may be approximated proximal of the passageway and the blood filter element. As another alternative, sheath 110 and shaft 130 with nosecone 120 may be removed from the patient separately from one another. As yet another alternative, procedure device 100 may be removed from the blood filter element as a single unit.

In FIG. 6F, with procedure device 100 removed from blood filter element 60 and from the patient, passageway 70 substantially seals in a self-sealing fashion, such that emboli E are retained within the blood filter element and cannot pass through the passageway. As seen in FIG. 6F, retrieval catheter 200 is advanced over guidewire G and elongated member 32 of attachment element 30, such that the retrieval catheter is positioned just proximal of blood filter element 60. Catheter 200 optionally may comprise delivery catheter 20, or may comprise a guide catheter or a catheter of larger diameter than catheter 20.

As seen in FIG. 6G, continued advancement of the catheter 200 relative to the blood filter element 60, or retraction of the blood filter element 60 relative to catheter 200, causes the blood filter element to collapse for retrieval within the catheter. Optional additional attachment wires 34 as illustratively depicted in FIG. 1 or 6C, may serve as recapture guide elements that provide a smooth transition during placement of catheter 200 over blood filter element 60. Captured emboli E are retained within closed portion 64 of the blood filter element during recapture of the filter element. Once recaptured, apparatus 10 is removed from the patient to complete the procedure. The guidewire may be removed at this time or used to facilitate additional procedures.

Referring now to FIG. 7 in combination with FIGS. 1-4 and 6, a method of using embolic protection apparatus 10 to protect against embolization during endovascular heart valve replacement is described. As seen in FIG. 7, blood filter element 60 has been deployed within a patient's aortic arch AA. The blood filter element contacts and substantially seals against a wall of the aorta, such that blood flowing through the aorta passes through the filter element. Apparatus 10 has been advanced in a retrograde fashion, such that opening 62 of the filter element faces the direction of blood flow through aortic valve AV and aortic arch AA.

Procedure device 100′ has been advanced through accommodating passageway 70 to the aortic valve. Catheter sheath 110 has been retracted, and replacement valve apparatus 150 has been deployed across the native aortic valve, e.g., via deployment elements 132 extending from central shaft 130′. Applicant has previously described endovascular heart valve replacement apparatus, for example, in co-pending U.S. patent applications Ser. No. 10/746,280, filed Dec. 23, 2003 and Ser. No. 10/870,340, filed May 16, 2004, which are incorporated herein by reference in their entirety.

Emboli E generated during deployment of apparatus 150 are filtered from the patient's blood stream via filter element 60. Procedure device 100′, apparatus 10 and the captured emboli then may be removed from the patient, as described previously with respect to FIG. 6. Apparatus 150 is left in place within the patient as an endovascular replacement of the patient's native valve.

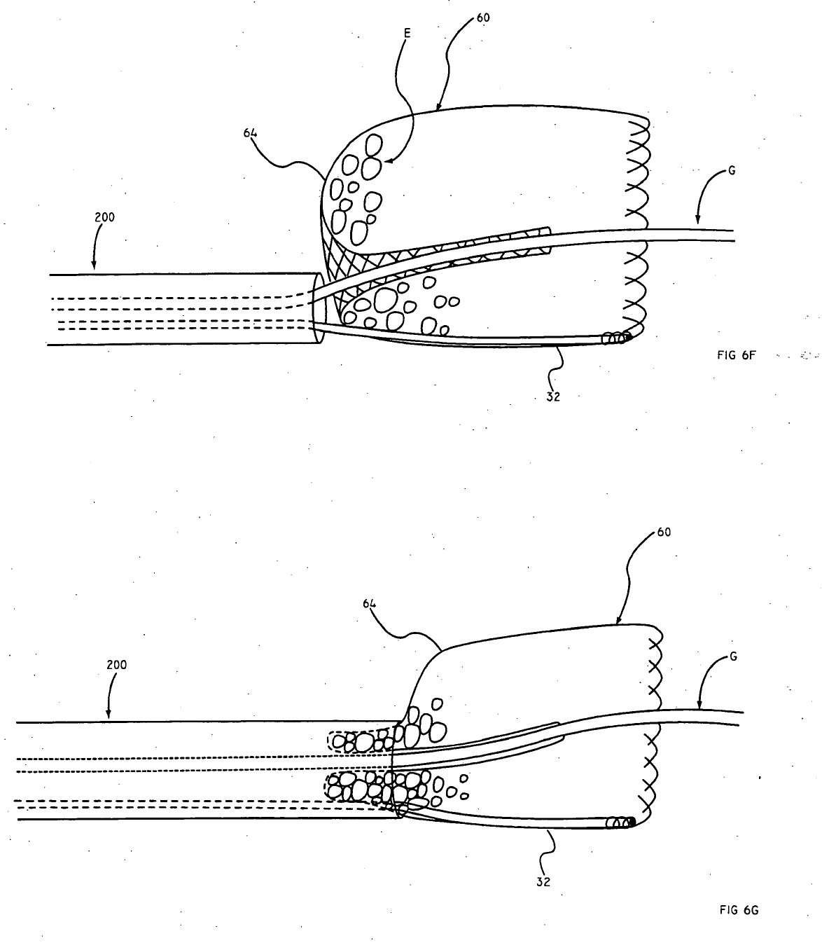

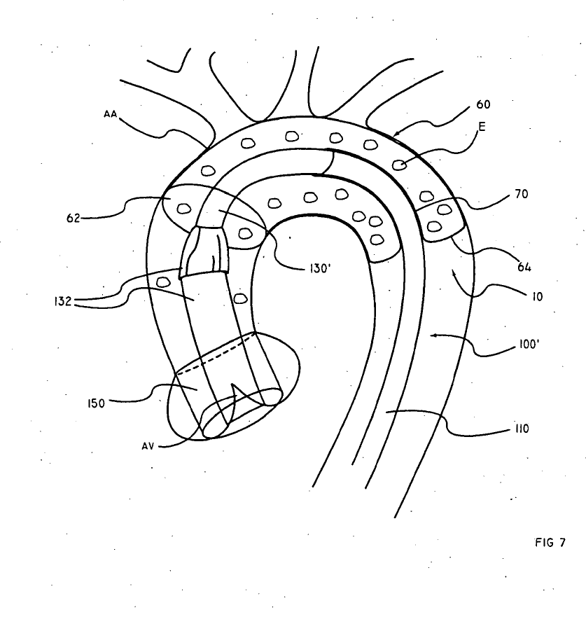

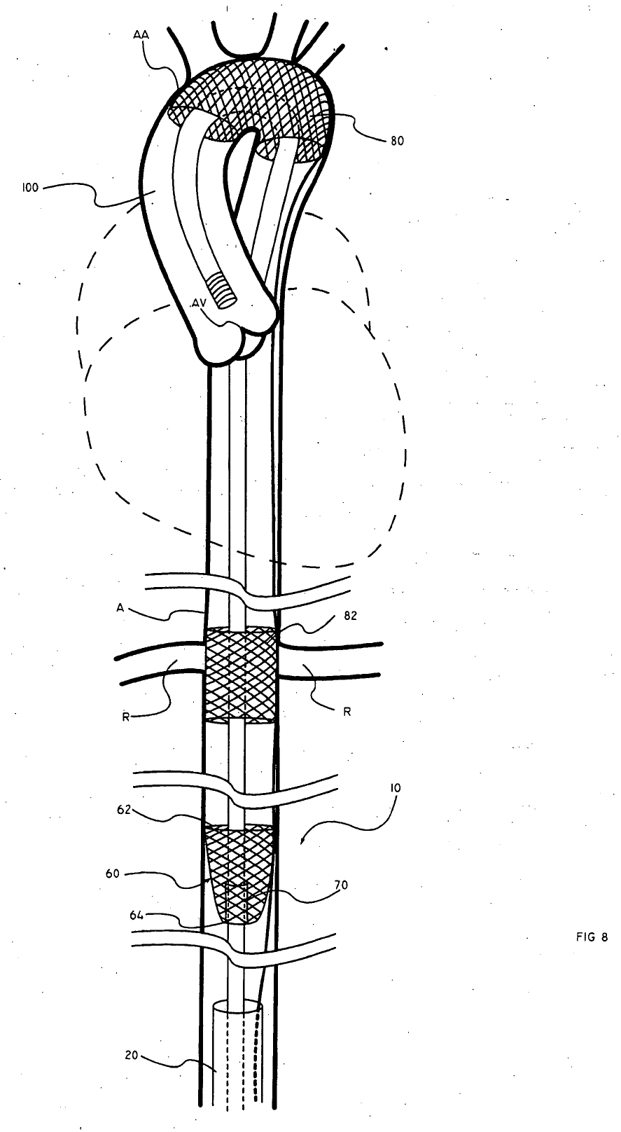

With reference to FIG. 8, another method of using the embolic protection apparatus to protect against embolization during endovascular heart valve replacement, valvuloplasty, etc., is described. In FIG. 8, blood filter element 60 has been deployed in conjunction with diverters 80 and 82 and more proximally within the patient's aorta A then as depicted in FIG. 7. Cerebral diverter 80 has been placed in the patient's aortic arch AA to divert embolic material away from cerebral vasculature, while renal diverter 82 has been placed in the patient's aorta across renal arteries R to divert embolic material away from the patient's kidneys. As will be apparent, filter element 60 alternatively may be positioned distal of the renal arteries, thereby obviating a need for renal diverter 82. The diverters and the blood filter may be combined in one delivery system as shown, with one “anchor/attachment” wire, or can be delivered by separate delivery systems (not shown). Applicant previously has described diverters for use during endovascular heart valve replacement, for example, in co-pending U.S. patent application Ser. No. 10/920,736, filed Aug. 17, 2004.

Procedure device 100, which may, for example, comprise endovascular heart valve replacement device 100′ of FIG. 7 or may comprise a valvuloplasty catheter, has been advanced through accommodating passageway 70 and through diverters 80 and 82 into proximity with aortic valve AV. Emboli generated during an endovascular procedure performed with device 100 on or near the aortic valve are carried downstream by blood flow and diverted by the diverters to blood filter element 60, which filters the embolic particles and captures them for removal.

With reference now to FIG. 9, an embodiment of embolic protection apparatus 10 is described comprising an alternative recapture guide element. In FIG. 9, apparatus 10 comprises recapture guide element 36 in place of (or in addition to some or all of) attachment wires 34. Element 36 extends from elongated member 32 of attachment element 30 and forms a loop about blood filter element 60. As seen in FIG. 9B and FIG. 10, as catheter 20 or retrieval catheter 200 is advanced relative to the blood filter element, recapture guide element 36 provides a transition that smoothly guides the catheter over the filter element while collapsing the filter element within the catheter.

Referring now to FIG. 11, apparatus 10 optionally may comprise a capture tool for recapturing the embolic protection apparatus. In FIG. 11, elongated member 32′ of attachment element 30′ comprises a tube having a lumen. One or more attachment wires 36 extend through the lumen and exit the elongated member through port(s) 33 formed in the elongated member at or near blood filter element 60. Each attachment wire 36 forms a loop or lasso about blood filter element 60 that terminates at a knot 37. Optionally, one or more attachment wires can be terminated at the same or a single knot 37. Knot 37 optionally might be a slip-knot.

The loop(s) provide a capture tool for collapsing filter element 60. The wires optionally may be independently controlled to collapse the filter element in sections. For example, opening 62 of filter element 60 may be closed with the distal-most lasso or loop to seal captured emboli within the filter element. Progressively more proximal lassos then may be actuated to facilitate recapture of the filter element within a sheath or catheter. Proximal control elements, such as clips, spacers or locks, may maintain desired diameter(s) of the lassos or loops to close or seal the filter element at a desired level.

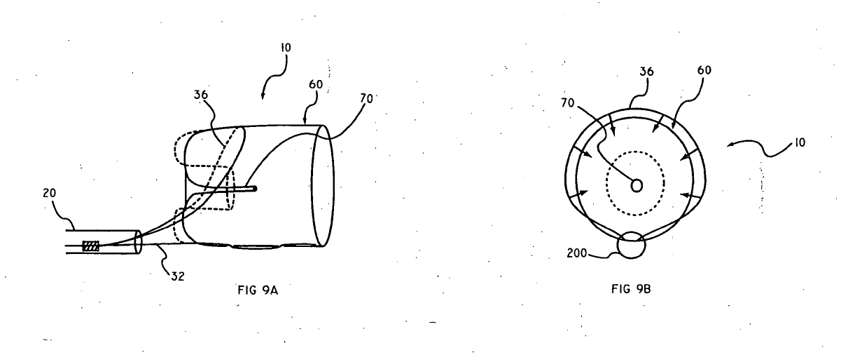

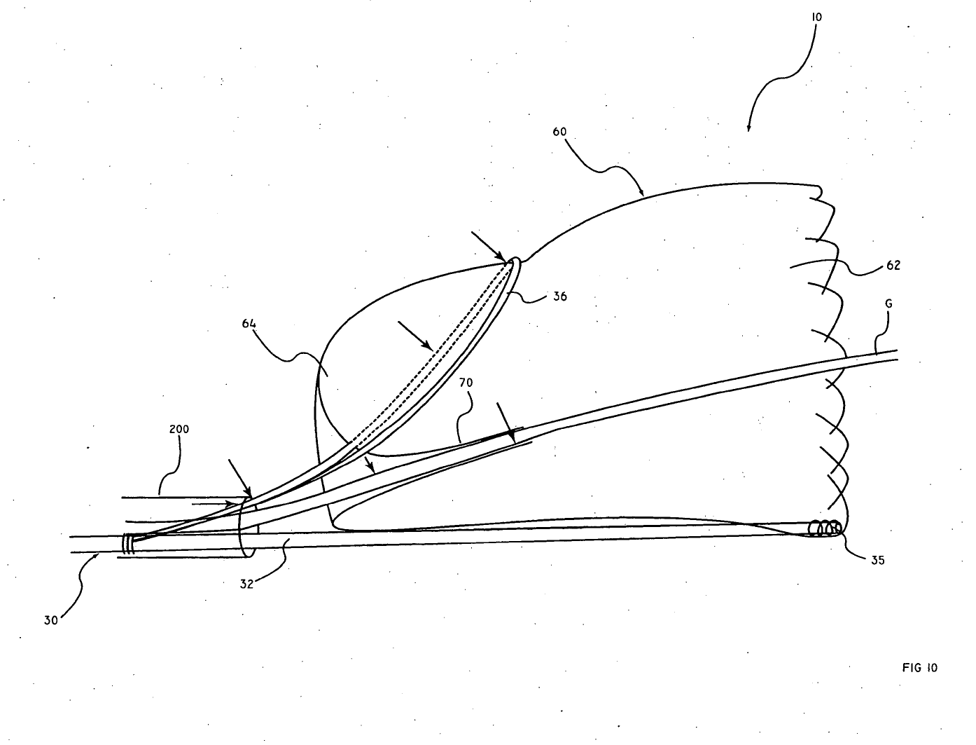

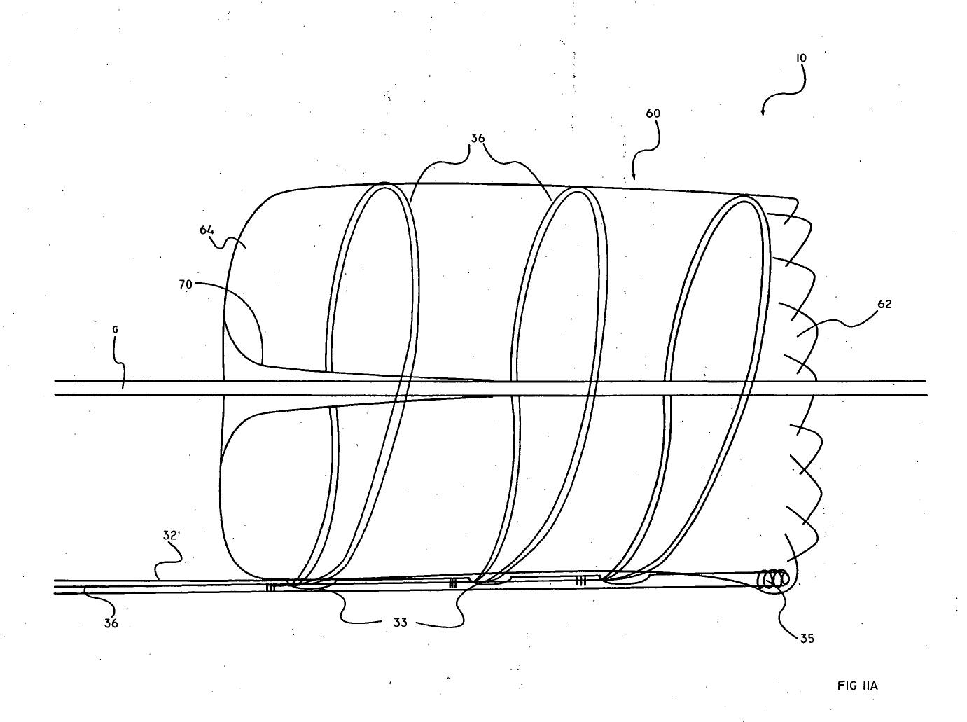

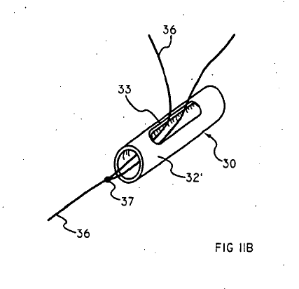

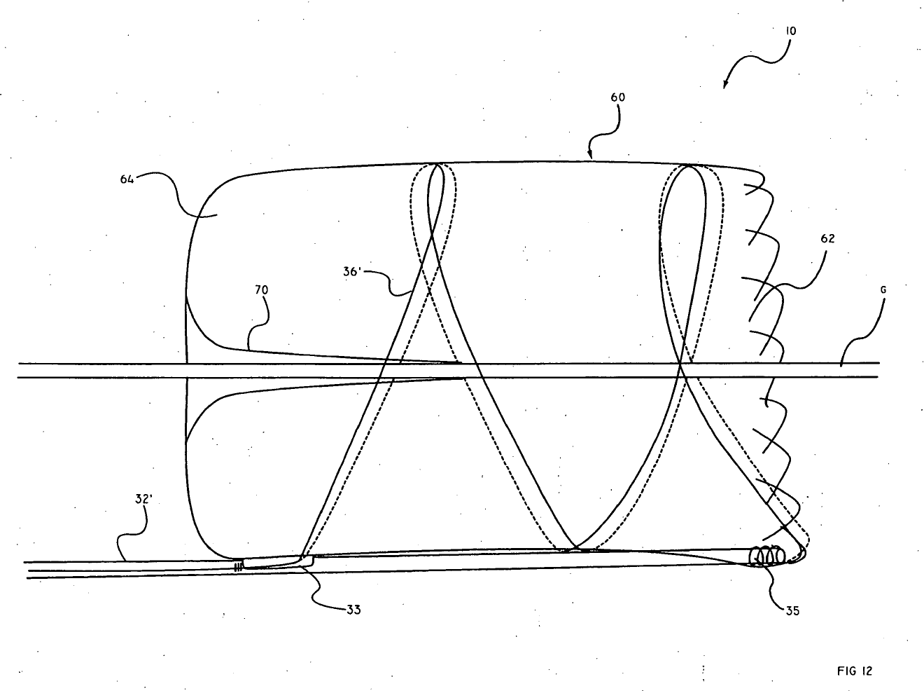

With reference to FIG. 12, an alternative embodiment of the capture tool of FIG. 11 is described. In FIG. 12, attachment wire 36′ comprises a spiral recapture wire that forms multiple loops about filter element 60. The attachment wire capture tool may be retracted to collapse the filter element for sheathing and/or recapture or retrieval.

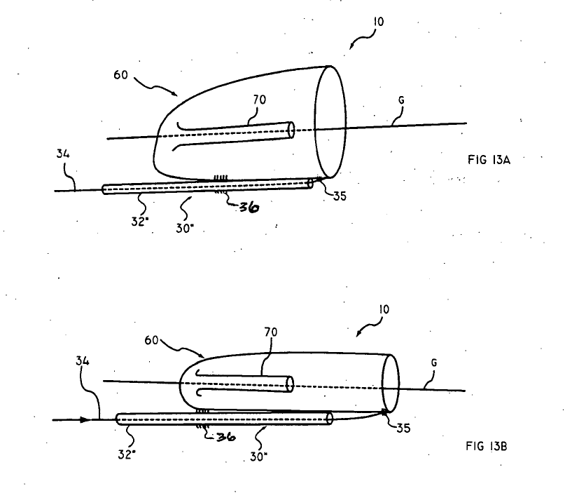

Referring to FIG. 13, another alternative capture tool is described. In FIG. 13, elongated element 32″ of attachment element 30″ comprises a tube having a lumen that is attached to a proximal region of filter element 60. Attachment wire 34 extends through the elongated member and attaches to a more distal region 35 of the filter element. Elongated member 32″ interfaces with filter element 60 at attachment point 36 at a more proximal location than the attachment of wire 34. As seen in FIG. 13B, by moving attachment wire 34 distally relative to elongated member 32″ (and/or by moving the elongated member proximally relative to the attachment wire) filter element 60 is longitudinally elongated and radially collapsed. This motion provides the filter element with a reduced profile that may facilitate sheathing and/or recapture of the filter element.

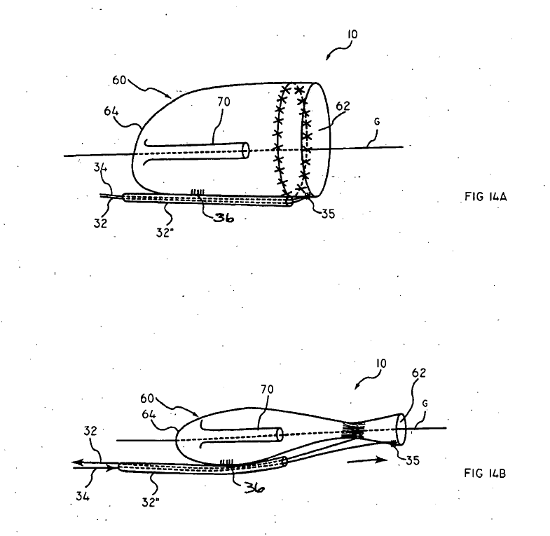

Referring to FIG. 14, an embodiment of apparatus 10 is described comprising the capture tool of FIG. 13 and a cinch mechanism for sealing the filter element thereby retaining captured particles within closed portion 64 of embolic protection apparatus 10. In FIG. 14, elongated member 34 forms a loop about, and/or is woven or braided within, filter element 60. Elongated member 32″ and attachment wire 32 facilitate longitudinal elongation and radial collapse of the filter element, while elongated member 34 facilitates cinching of the filter element. Cinching may facilitate retaining of captured particles, while elongation may facilitate recapture.

With reference now to FIG. 15, embodiments of embolic protection apparatus 10 are described having passageways 70 positioned at different locations along a diameter of the apparatus. In FIG. 15, filter elements 60 are bilaterally synmmetrical, but may not be radially symmetrical. Closed portions 64 of the filters comprise a radially asymmetric taper that extends, for example, from the attachment side of the filter element (the side on which attachment element 30 attaches to the filter element). The taper (angled proximal face) may facilitate sheathing and/or recapture of the filter element.

The closed portions 64 of the filters may additionally be configured such that they do not comprise a taper angle.

In FIG. 15A, passageway 70 is disposed in the center of the filter element, as with previous embodiments. In FIG. 15B, the passageway is disposed on the longitudinally shorter side of the filter element, while in FIG. 15C the passageway is disposed on the longitudinally longer side of the filter element. Placing the passageway off-center may balance the volume of the material comprising the radially asymmetric filter element to facilitate sheathing of the filter element. Furthermore, placing the passageway off-center may provide the filter element with a more predictable shape in the deployed configuration. Furtherstill, an off-center passageway may be used to guide a procedure device to have a particular bias, for example around the curve or within the passageway of a vascular lumen when advanced through the lumen of the accommodating passageway. An off-center passageway also may facilitate recapture and/or retrieval of the filter element.

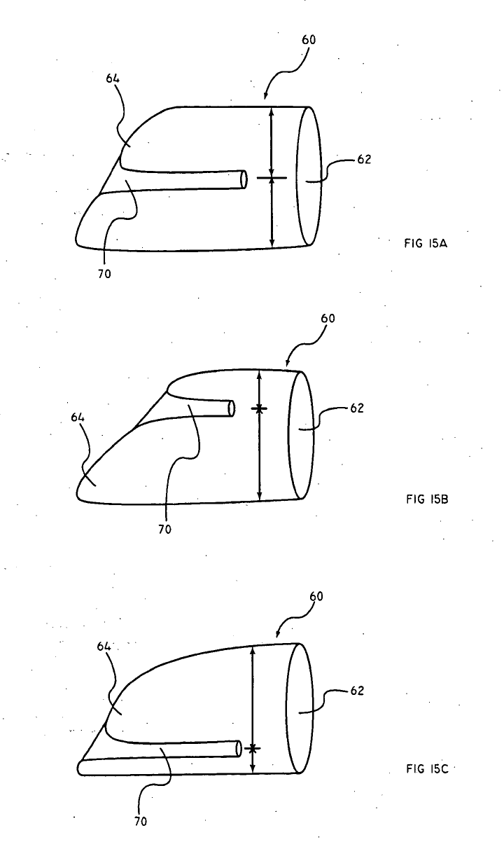

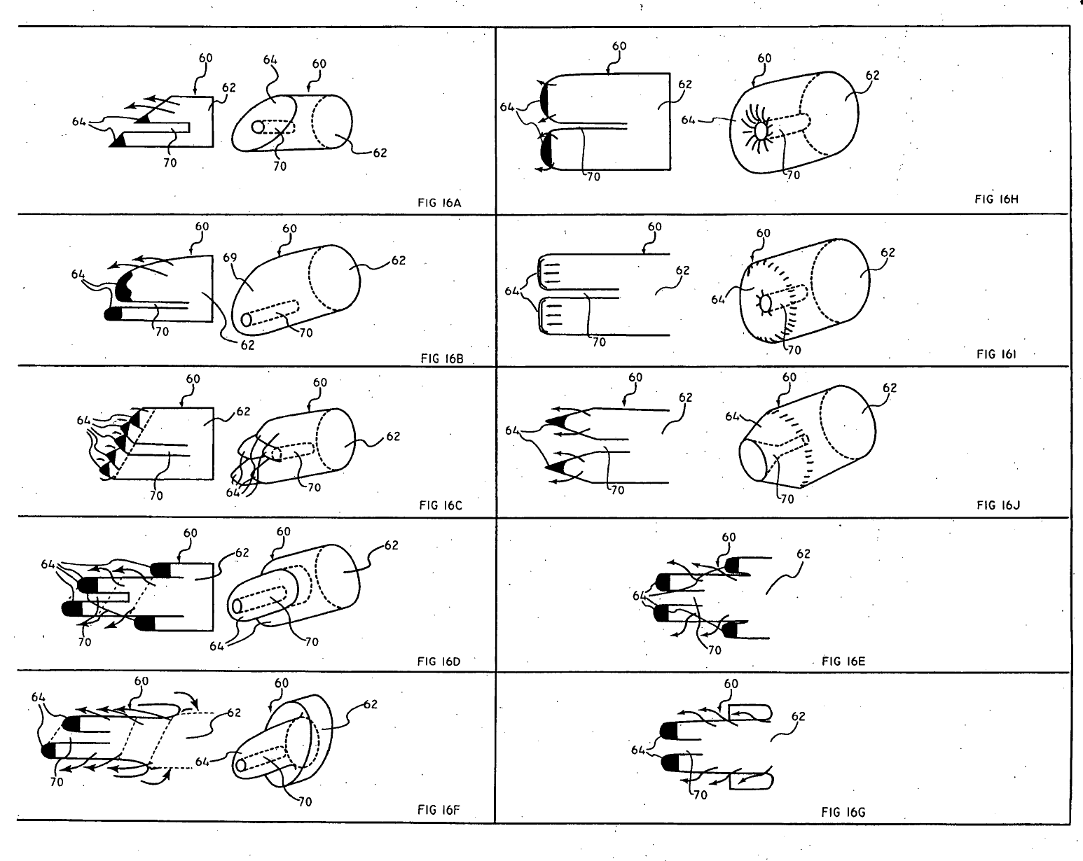

With reference now to FIG. 16, alternative embodiments of blood filter element 60 are described. These embodiments are provided for the sake of illustration and should in no way be construed as limiting. Darkened areas in FIG. 16 illustrate regions within the blood filter elements where embolic particles concentrate, while arrows indicate preferred paths for blood flow. As is well known, some prior filters cause occlusion after a period of time, as the emboli impede blood passage through the filter. These figures illustrate configurations are based on means to minimize the decrease in blood flow, caused by trapped emboli, by maximizing and varying the distribution of permeable surface area of the filter material.

FIG. 16A illustrates a variation of filter element 60 similar to the embodiment of FIG. 15A which comprises an angled proximal face. The embodiment of FIG. 16B comprises an off-center passageway 70, and closed portion 64 comprises a curved taper. FIG. 16C illustrates an embodiment comprising a plurality of closed conical portions 64 that form pockets for capturing emboli. In FIG. 16D, filter element 60 comprises a taper, as well as an inversion near opening 62 that provides the filter element with both a proximal closed portion and a distal closed portion for filtering and capturing emboli. The embodiment of FIG. 16E is similar to the embodiment of FIG. 16D, but is not tapered.

The embodiment of FIG. 16F comprises a taper, as well as a proximal eversion. This design may reduce the amount of material needed to form filter element 60 because the filter element forms a proximal seal against the wall of the blood vessel and then reduces in profile. This design may also facilitate delivery of the filter element in a reduced profile catheter. For example, the filter element may be collapsed for delivery with the eversion straightened, and the filter element may form the eversion during self-expansion to the deployed configuration. The embodiment of FIG. 16G is similar to the embodiment of FIG. 16F, but is not tapered.

In FIG. 16H, filter element 60 comprises rounded closed portion 64. In FIG. 16I, the closed portion is less rounded. In FIG. 16J, closed portion 64 is more conical.

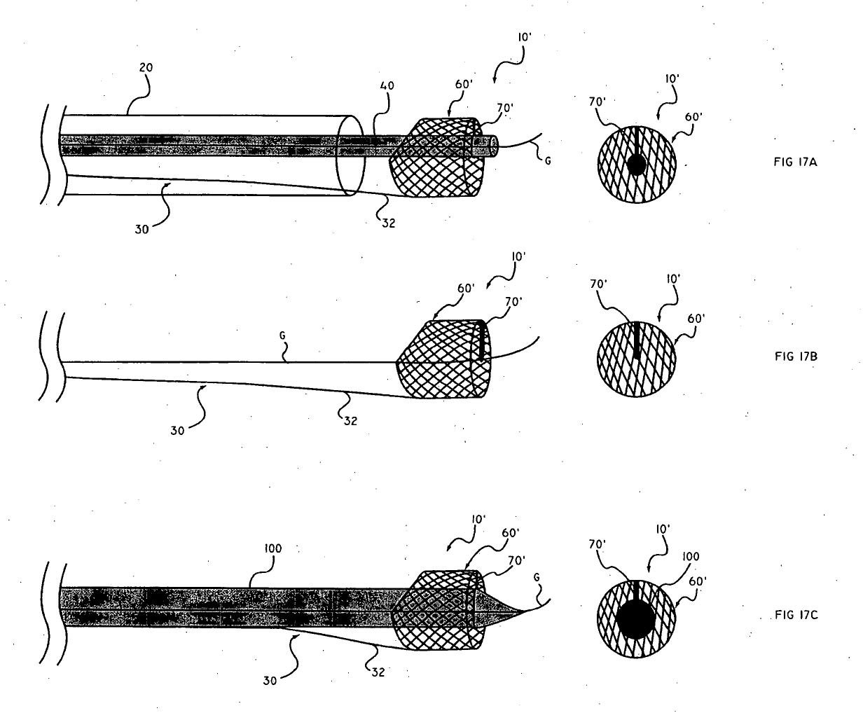

Referring now to FIG. 17, an alternative embodiment of the embolic protection apparatus is described having a passageway comprising a fold in the apparatus. Blood filter element 60′ of apparatus 10′ comprises passageway 70′, which is a slot or fold formed in the filter element. As seen in FIG. 17A, guidewire tube 40 is disposed through passageway 70′, and the passageway expands to accommodate the guidewire tube. As with passageway 70, passageway 70′ seals against the guidewire tube to reduce a risk of emboli passage through the passageway. In FIG. 17B, the guidewire tube is removed, and the passageway collapses and self-seals against guidewire G. As seen in FIG. 17C, procedure device 100 overcomes the sealing bias of passageway 70′ and is passed through the fold of filter element 60′. Passageway 70′ expands to accommodate the size and shape of the procedure device.

What is claimed is:

1. An intravascular embolic protection apparatus comprising:
a blood filter element adapted to capture particles and to allow blood to flow therethrough;
an opening adapted to face blood flow;
a closed portion adapted to retain captured particles; and
an accommodating passageway adapted to permit passage of a procedure device therethrough from a position proximal to the closed portion to a position distal to the opening and to substantially seal against passage of particles between the embolic protection apparatus and the procedure device by accommodating to a size and shape of the procedure device.

2. The intravascular embolic protection apparatus of claim 1, wherein the passageway is sealable to substantially prevent passage of particles through the passageway when a procedure device is not disposed in the passageway.

3. The intravascular embolic protection apparatus of claim 2, wherein the passageway is self-sealing.

4. The intravascular embolic protection apparatus of claim 3, wherein the passageway is biased toward a sealed position.

5. The intravascular embolic protection apparatus of claim 1, wherein the passageway has a tapered opening for catheter guidance.

6. The intravascular embolic protection apparatus of claim 1, wherein the passageway is expandable to permit devices of different sizes to pass through the passageway.

7. The intravascular embolic protection apparatus of claim 1, wherein the passageway is a lumen in the apparatus.

8. The intravascular embolic protection apparatus of claim 1, wherein the passageway is a fold in the apparatus.

9. The intravascular embolic protection apparatus of claim 1, wherein the apparatus is self-expanding from a delivery configuration to a deployed configuration.

10. The intravascular embolic protection apparatus of claim 9, wherein the passageway is expandable independent of the rest of the apparatus.

11. The intravascular embolic protection apparatus of claim 1, wherein the apparatus is radially symmetrical.

12. The intravascular embolic protection apparatus of claim 1, wherein the apparatus is bilaterally symmetrical.

13. The intravascular embolic protection apparatus of claim 1, wherein the passageway is located in a center of the apparatus.

14. The intravascular embolic protection apparatus of claim 1, wherein the passageway is located off-center of the apparatus.

15. The intravascular embolic protection apparatus of claim 1, wherein the closed portion defines a plurality of pockets adapted to trap and retain particles.

16. The intravascular embolic protection apparatus of claim 1, wherein the closed portion is tapered to facilitate recapture of the apparatus.

17. The intravascular embolic protection apparatus of claim 1, wherein the blood filter element defines the opening and the closed portion.

18. The intravascular embolic protection apparatus of claim 17, wherein the blood filter element defines the passageway.

19. The intravascular embolic protection apparatus of claim 18, wherein the opening surrounds the passageway.

20. The intravascular embolic protection apparatus of claim 18, wherein the closed portion surrounds the passageway.

21. The intravascular embolic protection apparatus of claim 18, wherein the blood filter element provides a bias force to substantially seal the passageway, and wherein the bias force may be overcome to permit passage of the procedure device through the passageway.

22. The intravascular embolic protection apparatus of claim 1, wherein the blood filter element comprises a mesh material.

23. The intravascular embolic protection apparatus of claim 22, wherein the mesh material is formed from a single wire.

24. The intravascular embolic protection apparatus of claim 22, wherein the mesh-material is formed from multiple wires.

25. The intravascular embolic protection apparatus of claim 22, wherein the mesh material is formed from multiple meshes.

26. The intravascular embolic protection apparatus of claim 22, wherein the mesh material is covered at least in part by filter material.

27. The intravascular embolic protection apparatus of claim 22, wherein the mesh material defines the passageway.

28. The intravascular embolic protection apparatus of claim 27, wherein the mesh material provides a bias force to substantially seal the passageway, and wherein the bias force may be overcome to permit passage of the procedure device through the passageway.

29. The intravascular embolic protection apparatus of claim 1, wherein the apparatus is adapted to be delivered via a catheter.

30. The intravascular embolic protection apparatus of claim 29, wherein the apparatus is further adapted to be delivered over a guidewire.

31. The intravascular embolic protection apparatus of claim 30 further comprising a guidewire tube.

32. The intravascular embolic protection apparatus of claim 29, wherein the apparatus further comprises an anchor element.

33. The intravascular embolic protection apparatus of claim 29, wherein the apparatus further comprises multiple attachment wires.

34. The intravascular embolic protection apparatus of claim 33, wherein the multiple attachment wires can be independently controlled.

35. The intravascular embolic protection apparatus of claim 29, wherein the apparatus is configured for delivery against blood flow.

36. The intravascular embolic protection apparatus of claim 1, wherein the apparatus is further adapted to be recaptured into a catheter.

37. The intravascular embolic protection apparatus of claim 36, wherein the blood filter element is further adapted to retain captured particles during recapture of the apparatus into the catheter.

38. The intravascular embolic protection apparatus of claim 36 further comprising a recapture guide element attached to the blood filter element.

39. The intravascular embolic protection apparatus of claim 38, wherein the recapture guide element comprises longitudinal recapture wires.

40. The intravascular embolic protection apparatus of claim 38, wherein the recapture guide element comprises spiral recapture wires.

41. The intravascular embolic protection apparatus of claim 36 further comprising a lasso for recapturing the apparatus within the catheter.

42. A method of performing an endovascular procedure on a patient with a procedure device, the method comprising:
delivering an embolic protection apparatus to a location within a vascular lumen of the patient, the embolic protection apparatus comprising an accommodating passageway;
passing the procedure device through the accommodating passageway from a point proximal to the embolic protection apparatus to a point distal to the embolic protection apparatus after the delivering step, the accommodating passageway accommodating to a size and shape of the procedure device;
performing the endovascular procedure; and
removing the procedure device from the patient.

43. The method of claim 42, wherein the embolic protection device comprises a filter, the method further comprising filtering blood flowing in the vascular lumen.

44. The method of claim 42, wherein delivering the embolic protection apparatus further comprises delivering the embolic protection device in a direction against the vascular lumen's blood flow direction.

45. The method of claim 42 further comprising removing the embolic protection apparatus from the patient.

46. The method of claim 45 further comprising retaining captured particles in the apparatus during the removing step.

47. The method of claim 45, wherein removing the embolic protection apparatus further comprises capturing the embolic protection apparatus in a catheter.

48. The method of claim 47, wherein capturing the embolic protection apparatus further comprises capturing the embolic protection apparatus with a capture tool.

49. The method of claim 42, wherein delivering the embolic protection apparatus further comprises permitting the embolic protection apparatus to self-expand.

51. The method of claim 42, wherein delivering the embolic protection apparatus further comprises delivering the embolic protection apparatus over a guidewire.

52. The method of claim 51, wherein the embolic protection apparatus further comprises a guidewire tube, the method further comprising removing the guidewire tube prior to the passing step.

53. The method of claim 42 further comprising anchoring the embolic protection apparatus with. an anchor element.

54. The method of claim 42, wherein passing the procedure device through the accommodating passageway further comprises opening the passageway.

55. The method of claim 54, wherein opening the passageway further comprises causing the passageway to self-seal against the procedure device.

56. The method of claim 54, wherein opening the passageway further comprises overcoming the passageway's sealing bias.

57. The method of claim 42, wherein the passageway comprises a lumen in the embolic protection apparatus, and wherein passing the procedure device through the accommodating passageway further comprises passing the procedure device through the lumen.

58. The method of claim 57, wherein passing the procedure device through the accommodating passageway further comprises using the passageway to guide the procedure device.

59. The method of claim 58, wherein the lumen of the accommodating passageway of the embolic protection apparatus is off-center.

59. The method of claim 42, wherein the passageway comprises a fold in the embolic protection apparatus, and wherein passing the procedure device through the accommodating passageway further comprises passing the procedure device through the fold.

60. The method of claim 42 further comprising substantially sealing the accommodating passageway after the removing step.
